(19) United States
(12) Patent Application Publication
(10) Pub. No.: US 2018/0279536 A1
(43) Pub. Date: Oct. 4, 2018

(54) GARDENING DEVICE FOR SOIL CULTIVATION AND METHOD FOR SOWING OR PLANTING WITH THE GARDENING DEVICE

(71) Applicant: Scheppach Fabrikation von Holzbearbeitungsmaschinen GmbH, Ichenhausen (DE)

(72) Inventor: Markus Bindhammer, Friedberg (DE)

(21) Appl. No.: 15/935,049

(22) Filed: Mar. 25, 2018

(30) Foreign Application Priority Data
Mar. 31, 2017 (EP) 17020129.7

(51) Int. Cl. A01B 1/02; A01B 79/00; G01N 33/24

(52) U.S. Cl. CPC A01B 1/022; G01N 2033/245; G01N 33/246; A01B 79/005

(57) ABSTRACT

A gardening device for soil cultivation and a sowing or planting method are provided. The gardening device includes a handle section, a ground contact section, a power supply, detecting devices for detecting variables corresponding to soil properties, a computing unit for calculating the soil properties from the number of variables corresponding to the soil properties, and an output device for outputting the soil properties. The ground contact section carries electrodes, via which the number of the variables corresponding to the soil properties are detected, and one of the number of detection devices is a nutrient detection device for detecting a number of variables corresponding to the nutrient content in the soil, the computing unit is designed for calculating the nutrient content in the soil from the number of variables corresponding to the nutrient content in the soil and the output device for outputting the nutrient content in the soil.

### CROSS REFERENCE TO RELATED APPLICATIONS

This application claims priority to European patent application EP 17 020 129.7, filed Mar. 31, 2017, the entire content of which is incorporated herein by reference.

### TECHNICAL FIELD

The invention relates to a gardening device for soil cultivation and to a method for sowing and planting performed with the help of such a gardening device.

### BACKGROUND

From document U.S. Pat. No. 8,836,504 a system is known which monitors a plant while growing. On the other hand, in order to check the soil in advance as to whether it is suitable for a particular plant, soil probes are already known, such as, the soil moisture measuring probe disclosed in German patent application DE 10 2012 106 841 A2. The probe described there has a capacitively operating sensor, which is located in a housing with a window recess. In the window recess sits an access element, which is impregnated with a hydrophilic material. Depending on how much water the surrounding soil has, the access element attracts much or little water and serves as the electricity medium for the capacitive measuring device. However, the soil moisture measuring probe is not only too expensive for the hobby gardener, but also too complicated to handle.

There are also already garden sensors that are introduced into the soil and there measure the moisture, light intensity and temperature and transmit this information, for example, to a user's cell phone, so that said user is informed about the currently prevailing conditions for sowing or planting, but also during the growth phase of the plants. While the horticultural success can be increased with such sensors, the handling remains tedious.

The trend is therefore to integrate the sensors already in the devices to be used by the gardener. E.g. such a device used by the gardener is disclosed by the U.S. Pat. No. 5,975,601 A, namely a gardening trowel made in one piece of a molten composite material, such as glass-reinforced nylon.

For example, international patent application WO 2016 118 000 A1 discloses a fertilizer application device with which holes can be pricked into the soil and the fertilizer can then be introduced therein. In addition, the device includes a temperature and a pH sensor to determine the temperature or pH of the soil before the fertilizer is introduced into the soil. However, such fertilizer application device is very expensive for horticultural use outside of industrial agriculture and is too specific in its actual application for the used measuring probes to be able to exploit their full utility.

By contrast, Canadian patent application CA 2 836 642 A1 shows a gardening trowel having a blade with a moisture sensor and a nitrate content sensor. On the blade handle, a display is provided in this case. Furthermore, the blade has a microchip, which can forward the measurement results to remote devices such as computers or mobile phones. The nitrate sensor is embedded in a silicate membrane and is located behind the blade, wherein diphenylamine reacts with nitrate particles in the silicate membrane and thereby turns blue and the sensor carries out a spectrophotometric detection of the coloration and thus the amount of nitrate. The moisture sensor is located on the front of the blade and consists of microscopic plates with a plurality of water pressure sensitive discs that swell when in contact with water and when, reaching a critical length, contact an actuator that emits a signal corresponding to the absorbed amount of water.

### SUMMARY

It is an object of the present invention to improve the structure of the gardening device so that high robustness is achieved at low cost and thus a sowing or planting procedure that can be carried out in an easy manner and with high reliability.

This object is achieved by a gardening device and a sowing or planting method as disclosed herein.

According to an aspect of the invention, the gardening device for soil cultivation has a handle section and a ground contact section attached thereto. Typically, the gardening device is designed as a gardening trowel, so that the ground contact section has the shape of a blade. Furthermore, the gardening device according to an aspect of the invention comprises a power supply, a number of detection devices for detecting a number of variables corresponding to soil properties, a computing unit for calculating the soil properties from the number of variables corresponding to the soil properties, and an output device for outputting the soil properties and/or the information based on the soil properties. The ground contact section carries electrodes, by which the number of variables corresponding to the soil properties is detected. Furthermore, the gardening device has a nutrient detection device for detecting a number of variables corresponding to the nutrient content in the soil.

The gardening device according to an aspect of the invention is characterized in that the ground contact section is made of plastic, typically made of fiber-reinforced plastic. Furthermore, the nutrient detection device is constructed so that it detects an electrical conductivity of the soil as a measure of the nutrient content. For this purpose, it comprises at least two electrodes, which are attached, spaced from each other, to the ground contact section of the gardening device. These two conductivity measuring electrodes can be used to measure the electrical conductivity of the soil, which is the greater the more nutrient ions are in the soil and therefore forms a measure of the nutrient content of the soil. The two conductivity electrodes consist of electroless nickel or comprise a layer of electroless nickel.

Due to the fact that the blade is made of a non-conductive material, the electrodes can be easily applied to the ground contact section without complex measures for the insulation of the individual electrodes from each other would have to be taken. Some plastics such as ABS also have a relatively high strength and high abrasion resistance, which is also well suited for use as a gardening device. This is particularly true, however, for fiber-reinforced plastics. Therefore, the ground contact section is made entirely of fiber-reinforced plastic, with glass-fiber-reinforced plastic in particular being well suited, since this also has a high dielectric strength in addition to high strength and abrasion resistance. It would also be conceivable to form only parts of the blade or of the ground contact section of plastic in the region of the electrodes. In particular, a layer consisting of a electroless nickel can be deposited very well on a ground contact section consisting of a plastic such as ABS, GRP or CFK.

Typically, the electrodes are attached to the surface of the ground contact section, e.g. of the blade, so that they can be brought into contact with the soil or the earth.

Another of the plurality of detection devices can be, for example, a moisture detection device for detecting a number of variables corresponding to soil moisture, wherein then the computing unit is set up for calculating the soil moisture from the number of variables corresponding to the soil moisture and the output device is suitable for outputting the soil moisture and/or soil-moisture-based information such as whether the soil moisture is suitable for a particular plant.

The high dielectric of the glass-fiber-reinforced plastic has a positive effect especially if, for example, the above-mentioned moisture detection device comprises two of the electrodes mounted spaced from each other on the ground contact section in the manner of a plate capacitor such that a capacitance of the plate capacitor is affected with earth as a dielectric when the earth is contacted with the ground contact section in the region between these capacitor electrodes. This achieves a cost-effective, but reliably working structure of the moisture detection device.

The capacitor electrodes of the moisture detection device may advantageously consist of a conductive material such as copper or a copper alloy and be applied to the ground contact section sealed against the environment in an air and moisture-proof manner. Therefore, the electrodes are not only inexpensive to produce, but also protected against corrosion or oxidation, so that the electrode material does not require expensive surface treatment or needs to consist of expensive elements or alloys. The seal also prevents the electrodes from coming into direct contact with the soil, thus avoiding unwanted current flow. It would also be possible to apply the two capacitor electrodes offset from each other on the front and back of the blade or the ground contact section. However, it would also be conceivable to embed the capacitor electrodes in the interior of the plastic or even to weave it into the fabric of the fiber reinforcement.

The two capacitor electrodes are advantageously located locally between the two conductivity measuring electrodes of the nutrient detection device, so that they can be arranged so close to one another that they can form a plate capacitor, whereas the distance of the two conductivity measuring electrodes of the nutrient detection device, which should allow a current flow between them, can be further apart.

Typically, each nickel layer of the conductivity measuring electrodes is covered in this case with a gold layer, which not only prevents the oxidation of the nickel, but can also produce a good conductive contact with the soil. Thus, the two conductivity measuring electrodes are particularly typically made of electroless nickel/immersion gold. The nickel layer may be, for example, between 4 and 7 μm thick and the gold layer mounted thereon between 0.05 and 0.1 μm.

The purpose of the gold layer is also to prevent the conductivity measuring electrodes, that is to say the nickel, from decomposing and releasing toxic ions for the plants.

The sowing or planting method according to an aspect of the invention may then include the following steps: determination of a favorable target value for soil moisture and nutrient content in the soil for a particular garden plant or vegetable species to be sowed or planted, at an area intended for sowing or planting, determining an actual value of the soil moisture and nutrient content in the soil, then comparing the target values with the actual values and, if the comparison is positive, sowing or planting the garden plant or vegetable species, wherein at least the determination of the actual value for the soil moisture is carried out with the aid of the gardening device according to an aspect of the invention, which is pushed into the soil at the intended sowing or planting point and which then, in the positive comparison case, can be used for producing the hole for the seeds or the plant during sowing or planting.

Advantageously, the computing unit is designed to calculate an output variable representing the soil moisture from the capacitance of the plate capacitor and/or from a quantity derived from the capacitance of the plate capacitor as an input variable. For this purpose, the computing unit may include a lookup table or the like which is stored in its memory, from which the context is apparent.

Depending on the thickness of the dielectric between the capacitor electrodes of the moisture detection device, i.e., depending on the soil moisture, this results in a different value for the capacitance of the plate capacitor formed by these two capacitor electrodes. The capacitance can be recorded. However, a vibration frequency as a measure of the capacitance of the plate capacitor or the dielectric of the earth and thus the soil moisture can be determined much easier and more accurately. Typically, the moisture detection device therefore has a vibration generator connected in series to the plate capacitor to form an oscillator circuit. The oscillator circuit can easily be evaluated if it outputs binary output signals as a tilting oscillator. For this purpose, the vibration generator can be designed as a Schmitt trigger. The frequency of the oscillation generated by contact with the soil can then be supplied instead of or in addition to the capacity of the plate capacitor to the computing unit as an input variable for the soil moisture.

Typically, the ground contact section is also interchangeable and in particular interchangeably mounted on the handle section without tools, for example via a bayonet lock or the connectors well-known from the garden area, e.g., from the company Gardena®. An additional connector or the like could be provided for the electrical contacting of the electrodes. The blade or the ground contact section configured as another working tool can then be easily replaced when worn, without having to renew the handle section containing the electronics.

Accordingly, the handle section typically comprises a handle, in which a microcontroller of the computing unit and a number of batteries of the power supply are housed, e.g. a 9-V block or a rechargeable battery, wherein on the handle an on/off switch and typically also a display of the output device is arranged.

Typically, the handle section has at its end facing away from the ground contact section a removable closure cap via which a battery receiving compartment inside the handle is accessible, so that the battery can be removed or replaced when it is depleted. The connection of the closure cap with the handle section is advantageously made waterproof, so that no dirt or water can get into the interior of the battery compartment. This of course applies to the further electronics, which are typically accommodated inside the handle in a separate chamber, such as the microcontroller of the computing unit, which chamber may also be tightly closed.

According to a further exemplary embodiment, a rechargeable accumulator could be provided as a battery, wherein the gardening device, at its handle section and there typically at its end facing away from the ground contact section, has a corresponding connection socket for a charger. Also conceivable would be a contactless battery charging system, in particular an inductive charging system in the manner of an electric toothbrush, so that then the battery could be permanently installed and sealed inside the handle.

In a further exemplary embodiment of the gardening device with a ground contact section interchangeably attached to the handle section, the gardening device has a plurality of differently shaped ground contact sections, each provided with the two capacitor electrodes and matching the handle section. The gardening device can then be used for versatile purposes. For example, one of the ground contact sections could be formed as a blade, another ground contact section as a trowel, and another ground contact section as a hoe, etc.

Further advantageously, the output device may include a communication module, with which data can be transferred wirelessly to an external device such as a smartphone or a PC. The communication module can be designed, for example, as a Bluetooth radio module or as a WLAN radio module and can be provided as an alternative or in addition to the display on the handle of the handle section. This not only provides a better overview of the determined measured values and the resulting consequences for sowing or planting, i.e., whether the place where the gardening device has been inserted into the soil is suitable or not for sowing or planting. Rather, the data can also be further processed on the external devices, or a user interface can be provided there in a simple manner, for example in the form of a software application, where the user can be provided with a selection of garden plants and/or vegetable species. This selection program can then continue to have, for example, favorable target values for soil moisture or nutrient content in the soil in the form of a database or look-up table, as well as a comparator device likewise typically designed as software, which compares the stored target values for the selected garden plant or vegetable with the detected actual values for the soil moisture and/or the nutrient content in the soil and then transmits the result for output to the output device or to the external device.

The gardening device may also have the user interface locally in the form of a touchscreen or in the form of selection keys or the like locally on the device itself, as well as the aforementioned selection program and the comparison device, e.g., in the form of program routines running on the microcontroller of the computing unit. For this purpose, the gardening device could contain a memory such as a micro SD card. However, as stated, it would also be conceivable to outsource the user interface and the associated software alternatively or additionally, in whole or in part, as part of the gardening device to an external device.

Furthermore, the gardening device can additionally be designed to detect further parameters influencing the sowing or planting. In particular, the gardening device could have a temperature detecting device for detecting a number of ambient temperatures of corresponding sizes, i.e., a temperature sensor for example in the form of a digital thermometer, which is connected for example via a bus connection to the microcontroller. Also conceivable would be other sensors that measure, for example, the color temperature of the ambient light, the pH of the soil or the air flow.

Furthermore, the gardening device can have a light detection device for detecting a number of variables corresponding to the light conditions in the environment, for example in the form of a phototransistor, which measures an illuminance of the incident light and which can likewise be connected to the microcontroller. The microcontroller can then measure the illuminance from the luminous flux. It would also be conceivable to design the light detection device as a solar cell.

Furthermore, the gardening device may have a clock device for determining the season, since the sowing or planting depends largely on sowing or planting at the right time of the year.

If the gardening device is supplemented by these additional facilities mentioned above, it is understood that the sowing or planting method according to an aspect of the invention can be further refined, wherein not only favorable target values for soil moisture and nutrient content in the soil are determined, but also for the ambient temperature, the lighting conditions in the environment and/or the sowing or planting time, which can then be compared with the determined actual values in order to be able to specify even more precisely whether the sowing or the planting is to be carried out.

### BRIEF DESCRIPTION OF THE DRAWINGS

The invention will now be described with reference to the drawings wherein:

FIG. 1 shows a perspective view of a garden trowel according to an exemplary embodiment of the invention;

FIG. 2 shows an exploded view of the garden trowel shown in FIG. 1;

FIG. 3 shows a further exploded view of the garden trowel shown in FIG. 1; and

FIG. 4A shows a first circuit diagram of the garden trowel shown in FIGS. 1-3.

FIG. 4B shows a second circuit diagram of the garden trowel shown in FIGS. 1-3.

FIG. 4C shows a third circuit diagram of the garden trowel shown in FIGS. 1-3.

FIG. 5A shows a fourth circuit diagram of the garden trowel shown in FIGS. 1-3.

FIG. 5B shows a fifth circuit diagram of the garden trowel shown in FIGS. 1-3.

FIG. 6A shows a sixth circuit diagram of the garden trowel shown in FIGS. 1-3.

FIG. 6B shows a seventh circuit diagram of the garden trowel shown in FIGS. 1-3.

FIG. 6C shows an eighth circuit diagram of the garden trowel shown in FIGS. 1-3.

FIG. 6D shows a ninth circuit diagram of the garden trowel shown in FIGS. 1-3.

### DESCRIPTION OF EXEMPLARY EMBODIMENTS

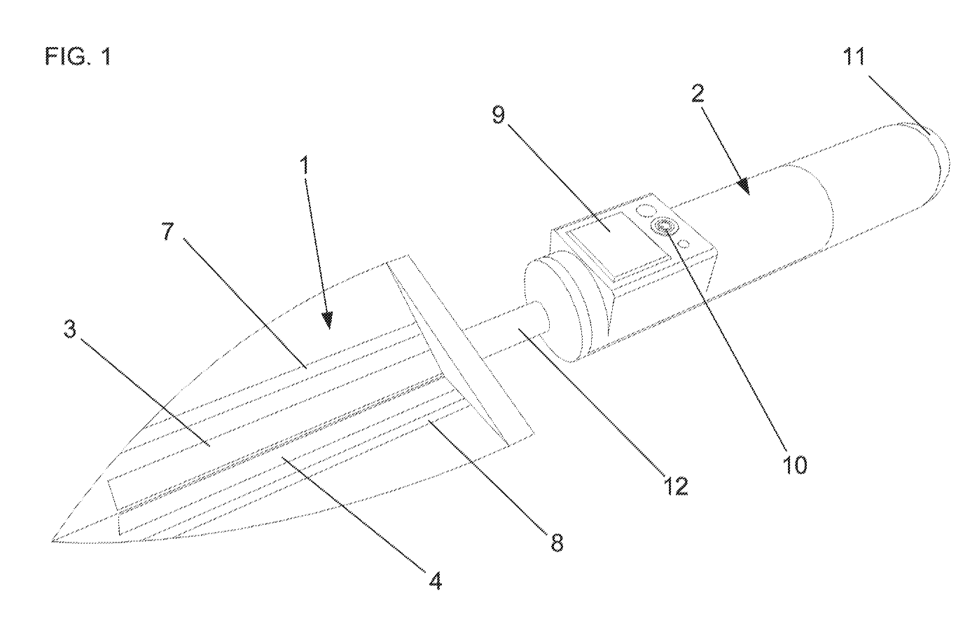

FIG. 1 shows a garden trowel with which it is possible to measure, while working, the moisture and the nutrient content of the soil or of the plant or potting soil and to determine the ambient temperature and the lighting conditions, so that one immediately receives indicators, whether the planting or seed stock is supplied at this point with sufficient water and nutrients and whether the light and temperature conditions are conducive to the planting or seed stock.

For this purpose, the electronic garden trowel is inserted at a selected location into the ground or earth. The electronic garden trowel now measures nutrient content and soil moisture, temperature, ambient light conditions and any other parameters and then informs the user via the display or other user interface based on the data and the current date of an internal real-time clock (not shown) whether or not the season and the selected location are suitable for the selected plant or vegetable species. If the selected location and season are suitable, the plant or seed can be introduced there.

The garden trowel has a blade 1, which is typically made of glass-fiber-reinforced plastic, since it is well suited as a dielectric and has high stability and abrasion resistance. On the upper side of the blade 1, four electrodes 3, 4, 7, 8 are applied. It would also be conceivable to attach the electrodes to the underside of the blade. The electrodes 3, 4, 7, 8 can be vapor-deposited, glued, printed or applied by other mechanical or chemical processes. Of the four electrodes 3, 4, 7, 8, two first electrodes 3, 4 designated as capacitor electrodes serve to measure the moisture of the soil. The two other outer electrodes 7, 8, which are designated as conductivity electrodes, are used to measure the nutrient content of the soil.

The more nutrient ions are in the soil, the greater the electrical conductivity. In order to avoid electrochemical corrosion of the conductivity measuring electrodes 7, 8, these two external conductivity measuring electrodes 7, 8 are typically made of electroless nickel/immersion gold (electroless nickel immersion gold, ENIG), a process which is already used today for printed conductors on printed circuit boards with a typical gold layer of 0.05-0.1 μm and nickel layer of 4-7 μm.

The immersion gold layer prevents oxidation of the nickel. This is intended to prevent the conductivity measuring electrodes 7, 8 from decomposing and releasing toxic ions for the plants.

The two inner capacitor electrodes 3, 4 form a plate capacitor. These two capacitor electrodes 3, 4 are sealed air-tight and moisture-tight over their entire surface in order to prevent the water or soil or air from being able to reach the electrode surfaces directly. They serve to determine the soil moisture. Since these capacitor electrodes 3, 4 are sealed and thereby not subject to corrosion or oxidation, their electrode material does not have to be surface-treated with difficulty or consist of expensive elements or alloys. For example, copper or the like can be used. The moist soil or the moist earth serves as a dielectric, which influences the capacitance of the plate capacitor formed from the two electrodes 3, 4.

Since the blade 1 and especially the exposed outer electrodes 7, 8 are worn down by the gardening that can be performed therewith, the blade 1 is designed so that it can be replaced without special tools. This also opens up the possibility of using blades of different shapes and offering them as accessories.

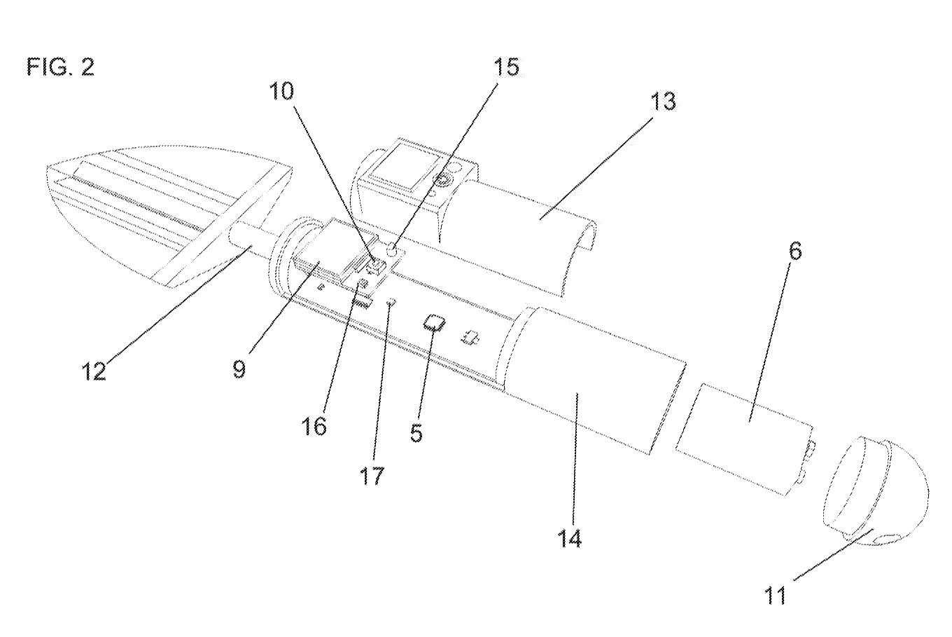

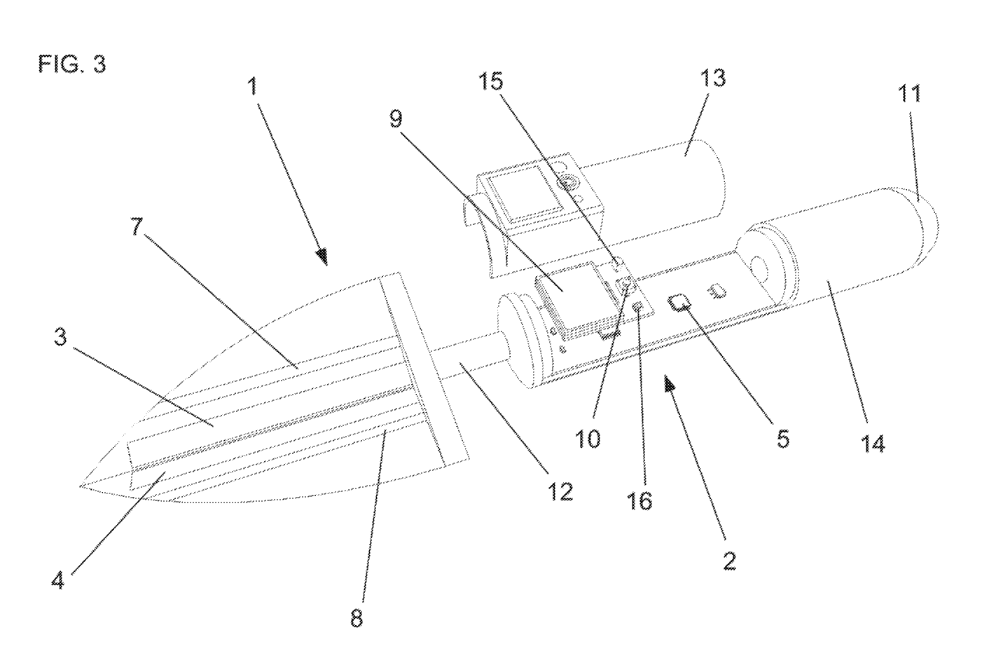

In addition to an ON/OFF switch 10, an OLED or LCD display 9 and a microcontroller 5, there are three further sensors in the garden trowel shown on or in a handle section 2 (see FIGS. 2 and 3)—a 3-axis acceleration sensor 17, a phototransistor 16, and a temperature sensor 15. The 3-axis acceleration sensor 17 measures the inclination of the electronic garden trowel. The analog or digital and pre-filtered values of the 3 axes are fed to a microcontroller 5, which determines the position of the electronic garden trowel.

Depending on the inclination, the values displayed on the OLED or LCD display 9 are rotated, so that the user can read the values no matter in which position the electronic gardening trowel is currently located. A similar principle can already be found today in most smartphones. In addition, it would be possible to use the 3-axis acceleration sensor 17 as a user interface.

The phototransistor 16 measures the illuminance, i.e., what fraction of the luminous flux arrives on a square meter surface of the illuminated object.

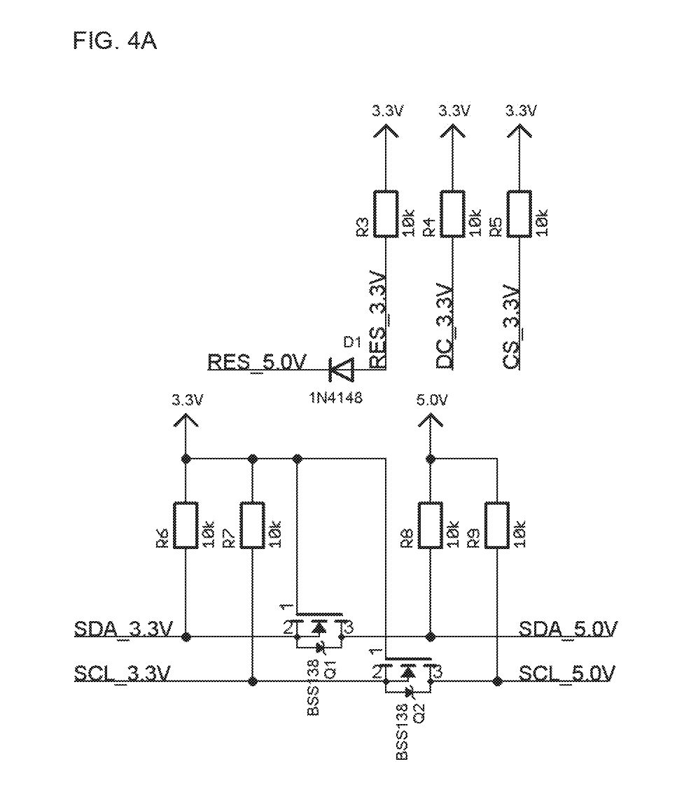

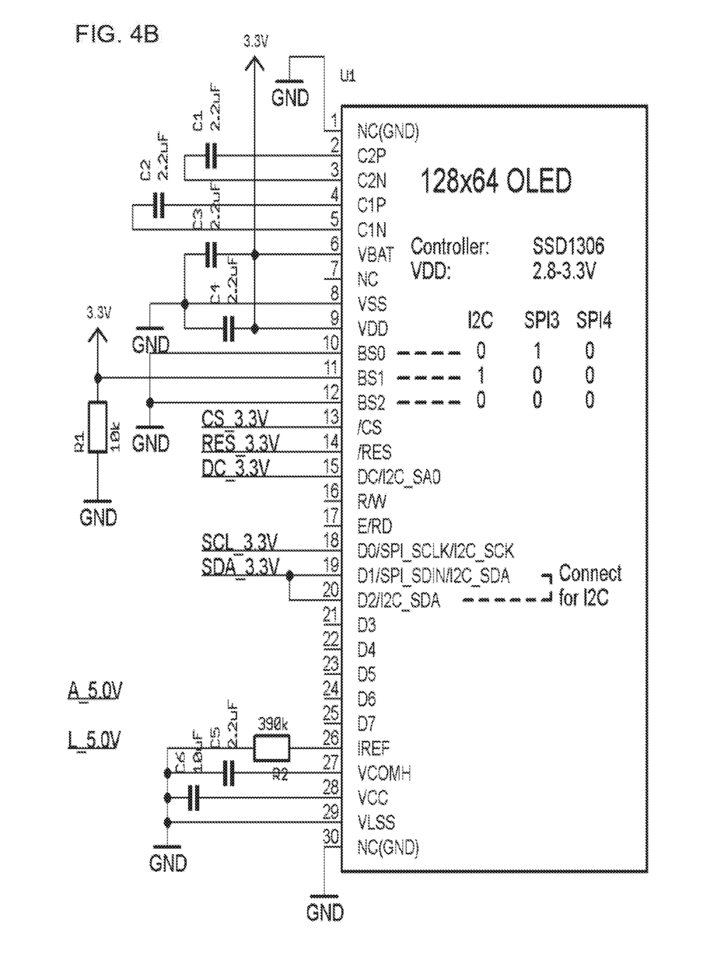

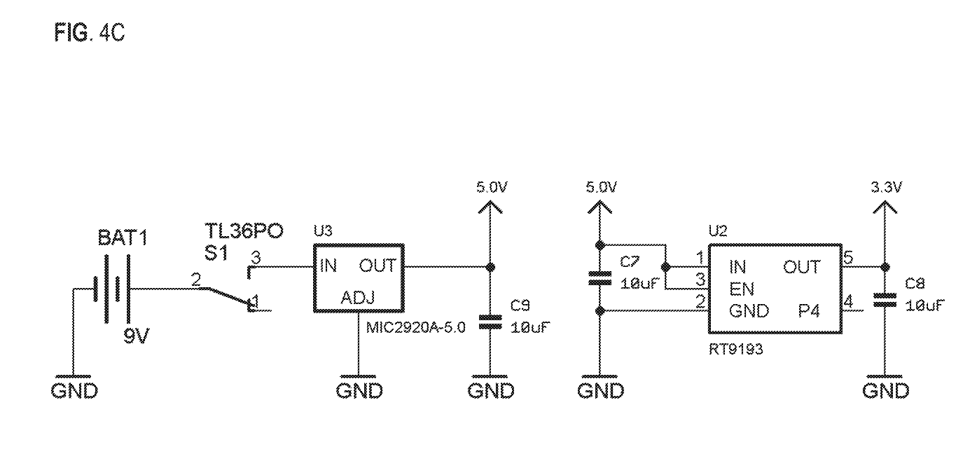

FIGS. 4A to 4C, 5A, 5B, and 6A to 6D show possible examples of circuit diagrams for the above-described garden trowel without the aforementioned internal real-time clock and user interface, which as mentioned may include, for example, buttons, a trackball, miniature joystick or touch screen.

FIGS. 4A to 4C show as an example of the display 9, a 128×64 OLED display, referred to here in the diagram as U1, which is configured so that the communication runs on an I²C BUS, also for the usual 5V microcontroller 5, referred to here in the circuit diagram as U5, the necessary level converters of the 3.3V I²C bus and the 3.3V reset line as well as the power supply for the complete circuit.

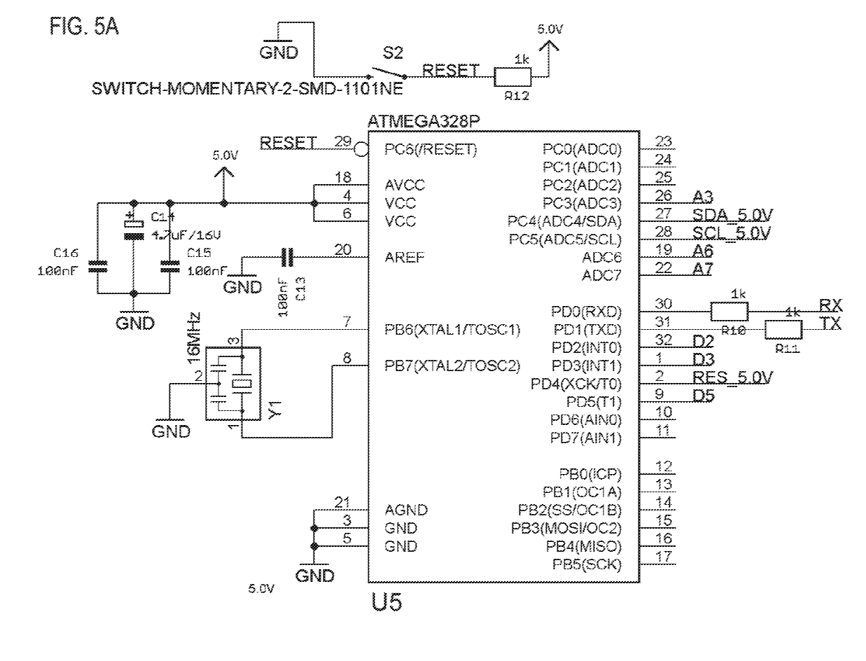

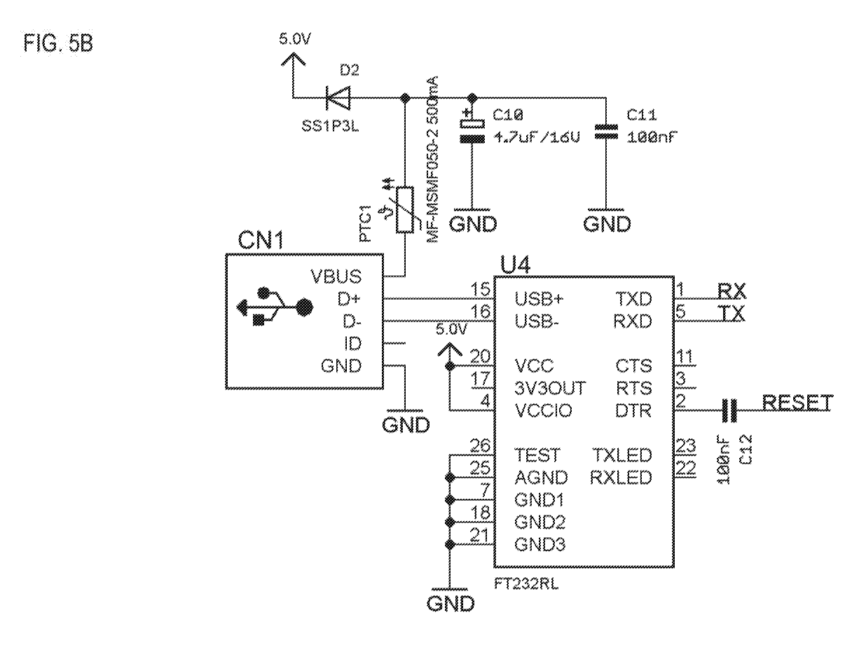

FIGS. 5A and 5B show as another example a typical 5V microcontroller U5 and its periphery. In FIGS. 5A and 5B, the microcontroller U5 has a USB interface made up of CN1 and the USB/serial converter U4 in order to program and debug the same. But this does not necessarily have to be the case. Programming and debugging of the microcontroller U5 could also be done via an SPI interface or the like, so that CN1 and U4 could then be omitted.

The plate capacitor comprising the capacitor electrodes 3, 4 (in the circuit diagram: PROBE 1, PROBE 2) forms, together with a Schmitt trigger IC2 of the type 74HC14, an oscillator and thus, in total, the moisture detection device. Depending on the area of the capacitor, the frequency is between a few 100 kHz and several MHz. The oscillator itself is an RC oscillator, wherein the one capacitor electrode is not at GND, as usual, but at signal level to minimize interference that might be spread over the ground line. The moister the soil or the earth, the greater the capacity of the capacitor and the lower the frequency of the oscillator. The output signal of the oscillator is supplied to a digital input of the microcontroller U5, which measures the frequency of the oscillator and calculates the soil moisture from it.

In addition to the two conductivity measuring electrodes 7, 8, which can be seen in the circuit diagram as PROBE3 and PROBE4, the nutrient detection device likewise comprises further electronic components. One of the two conductivity measuring electrodes 7, 8, or in the circuit diagram PROBE3 or PROBE4, is connected to the base of an npn transistor Q5, the other via a series resistor R19 to the positive supply voltage. The more conductive the ground, the more the transistor Q5 conducts. The transistor Q5, which itself acts like a resistor, forms a voltage divider with the resistor at an emitter R17. The signal is fed to an analog input of the microcontroller 5, or in the circuit diagram to U5. This measures the voltage and calculates therefrom the nutrient content of the soil. To further increase the life of the PROBE 3, PROBE 4 electrodes, they are not permanently connected to the supply voltage. Via a p-channel MOSFET Q4, the sensor formed from the electrodes PROBE3 and PROBE4 is only activated by the microcontroller U5 if a command for measuring the nutrient content is given in the program sequence.

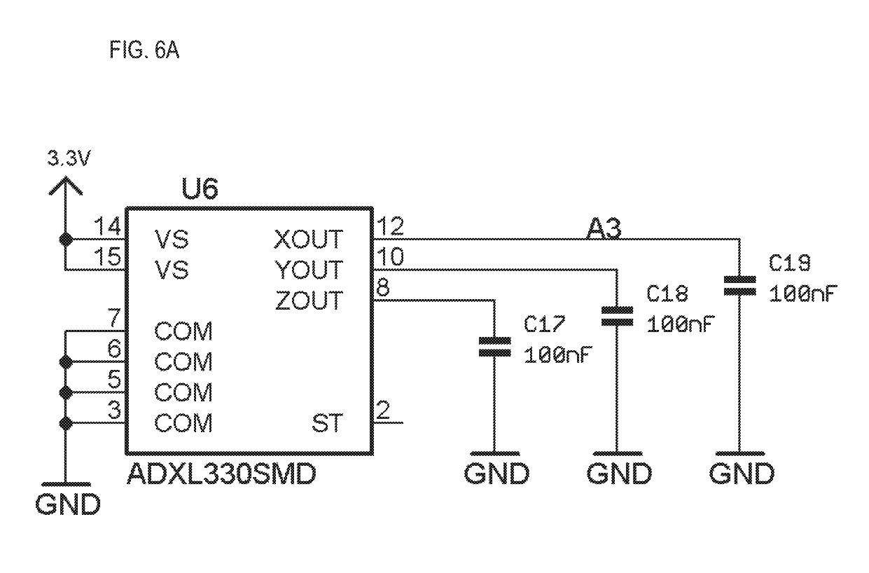

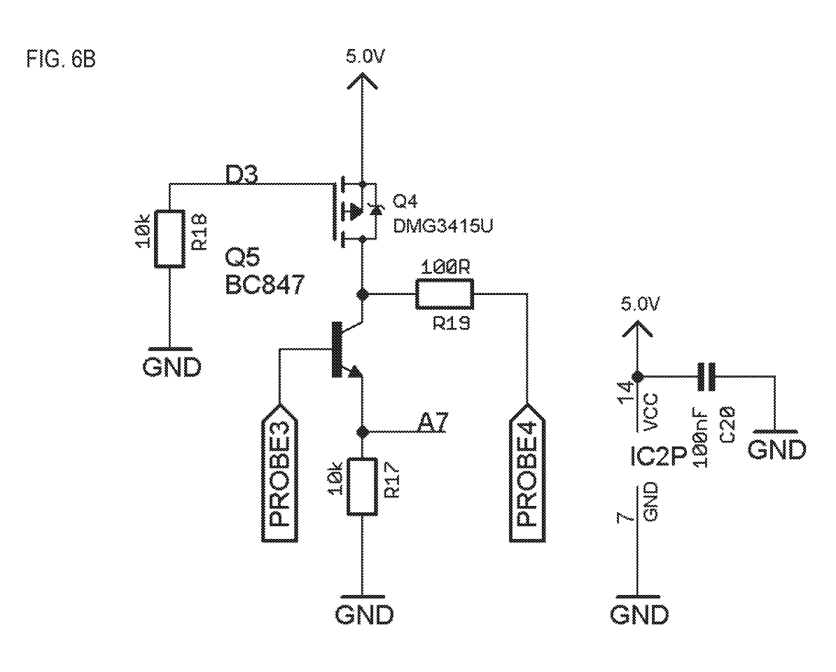

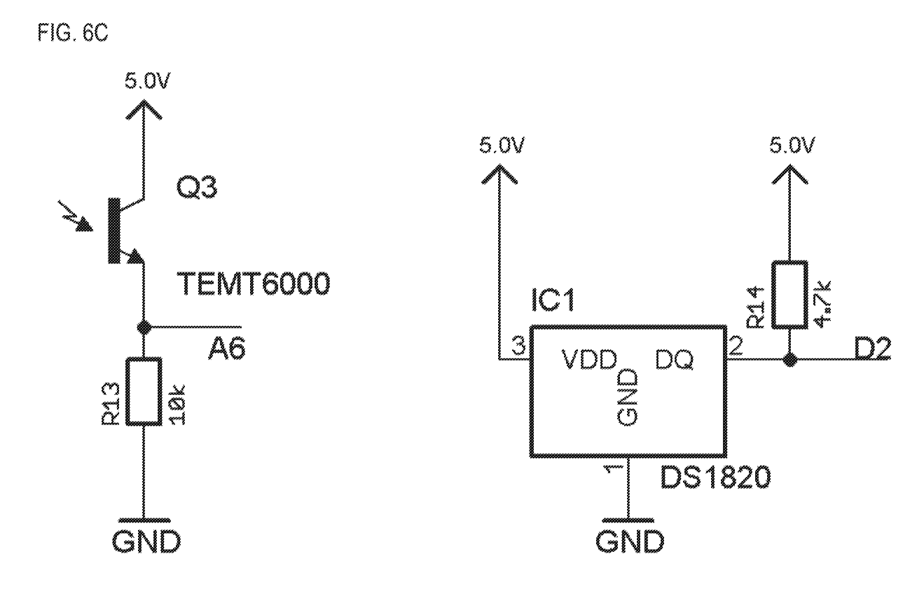

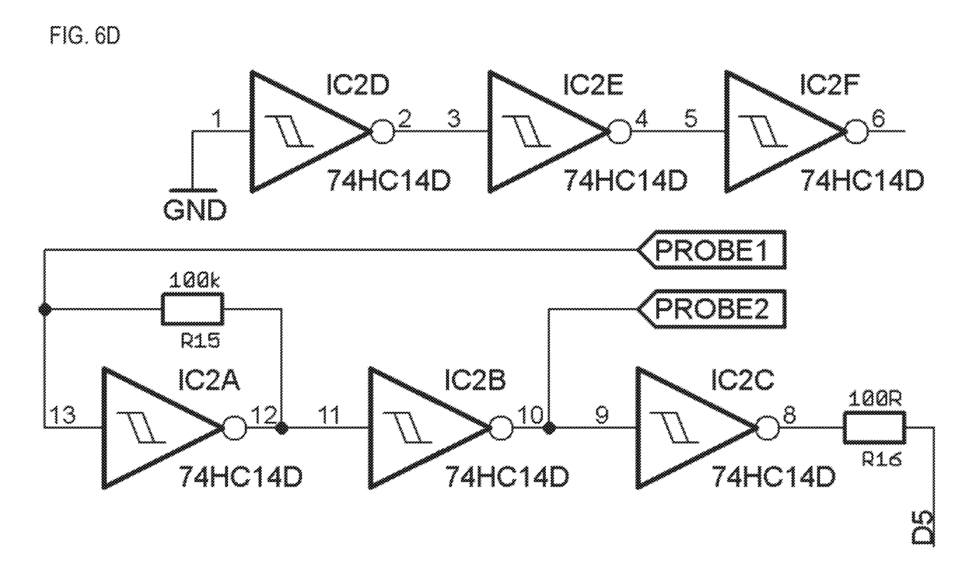

FIGS. 6A to 6D show once again the five sensors of the garden trowel: the 3-axis acceleration sensor 17 consisting of three low-pass filter capacitors C17 to C19, here in the circuit diagram U6, the ambient light sensor consisting of the phototransistor 16, here in the circuit diagram Q3 and the resistor R13, the temperature sensor 15 consisting of IC1 and R11, the nutrient sensor consisting of the further electrodes PROBE3 and PROBE4, the npn transistor Q5, the p-channel MOSFET Q5 and the resistors R17 to R19, and the capacitive moisture sensor consisting of the first electrodes PROBE1 and PROBE2, the Schmitt trigger IC2 and the resistors R15 to R16.

The phototransistor Q3 and the resistor R13 are connected as a voltage divider. The signal is fed to an analog input of the microcontroller U5. The greater the fraction of luminous flux, the greater the voltage at the output of the voltage divider. From the measured voltage, the microcontroller U5 then calculates the illuminance. The temperature sensor IC1 is, for example, as shown here, a digital thermometer with a programmable resolution of 9-12 bits, a measuring range of −55° C. to +125° C. and a tolerance of ±0.5° C. in the range of −10° C. to +85° C. The temperature sensor IC1 measures the ambient temperature and communicates with the microcontroller U5 via the so-called single-wire bus.

The electronic garden trowel is supplied by a standard 9V block battery 6, here BAT1 in the circuit diagram, or similar compact batteries or accumulators. The battery BAT1 can be removed and exchanged from the rear end of the handle 2 when it is depleted. For this purpose, the closure cap 3, which is provided with a thread or other sealing method, must first be removed. The cap 3 and the handle 2 themselves are waterproof, so that no water or dirt can get inside and damage the electronics.

It would also be conceivable not to have to remove the battery for charging. The electronic garden trowel would then require a corresponding socket for a charger or a contactless battery charging system, as it is already common today, for example, for electric toothbrushes.

The electronics are accommodated in a chamber separate from the battery compartment in the handle section 2, which has a closure cover 13 for this purpose. From this chamber lines are led to the blade 1, through a hollow connecting shaft 12 of the handle section. 2.

It is understood that the foregoing description is that of the exemplary embodiments of the invention and that various changes and modifications may be made thereto without departing from the spirit and scope of the invention as defined in the appended claims.

What is claimed is:

1. A gardening device for soil cultivation, comprising a handle section, a ground contact section attached thereto, for example a blade, a power supply, a number of detection devices for detecting a number of variables corresponding to soil properties, a computing unit for calculating the soil properties from the number of variables corresponding to the soil properties, and an output device for outputting the soil properties and/or soil properties based information, wherein the ground contact section carries electrodes, via which the number of the variables corresponding to the soil properties are detected, and wherein one of the number of detection devices is a nutrient detection device for detecting a number of variables corresponding to the nutrient content in the soil, and the computing unit is designed for calculating the nutrient content in the soil from the number of variables corresponding to the nutrient content in the soil and the output device for outputting the nutrient content in the soil, wherein the ground contact section is made of fiber-reinforced plastic such as glass-fiber-reinforced plastic, and the nutrient detection device detects an electrical conductivity of the soil as a measure of the nutrient content and comprises at least two of the electrodes for conductivity detection, which are mounted spaced apart on the ground contact section, wherein these conductivity measuring electrodes comprise or consist of a layer deposited as electroless nickel on the ground contact section.

2. The gardening device according to claim 1, wherein the electrodes are applied to the surface of the ground contact section.

3. The gardening device according to claim 1, wherein the gardening device has a moisture detection device for detecting a number of variables corresponding to soil moisture, wherein the computing unit is designed for calculating the soil moisture from the number of variables corresponding to the soil moisture and the output device for outputting the soil moisture and/or data based on the soil moisture, and wherein the moisture detection device has at least two of the electrodes, which are mounted as a plate capacitor spaced from each other on the ground contact section, such that a capacitance of the plate capacitor is influenced with the soil as a dielectric when the soil is contacted with the ground contact section in the region between these capacitor electrodes.

4. The gardening device according to claim 1, wherein the computing unit is designed to calculate an output variable representing the soil moisture from the capacitance of the plate capacitor and/or a variable derived from the capacitance of the plate capacitor as an input variable.

5. The gardening device according to claim 1, wherein the moisture detection device comprises a vibration generator, which is interconnected to the plate capacitor to form an oscillator circuit, and wherein the frequency of the generated vibration and/or the capacitance of the plate capacitor is supplied to the computing device as an input variable for the soil moisture.

6. The gardening device according to claim 1, wherein the two capacitor electrodes of the moisture detection device consist of a conductive material such as copper or a copper alloy and are applied to the ground contact section in an air-tight and moisture-tight sealed manner against the ambient environment.

7. The gardening device according to claim 1, wherein the ground contact section is interchangeably attached to the handle section.

8. The gardening device according to claim 1, wherein the handle section comprises a handle in which a microcontroller of the computing unit and a number of rechargeable batteries of the power supply are housed, wherein on the handle an ON/OFF switch and also a display of the output device are arranged.

9. The gardening device according to claim 7, wherein the gardening device comprises a number of differently shaped ground contact sections including a blade, a trowel, and a hoe, in each case provided with the two capacitor electrodes and matching the handle section.

10. The gardening device according to claim 1, wherein the gardening device has a temperature detecting device for detecting a number of variables corresponding to the ambient temperatures, a light detecting device for detecting a number of variables corresponding to the light conditions in the environment, and/or a clock device for determining the season, wherein the computing unit is configured for the ambient temperature, the lighting conditions in the environment and/or the season, and wherein the output device is configured for outputting the ambient temperature, the lighting conditions in the environment and/or the season.

11. The gardening device according to one of the claim 2, wherein the two capacitor electrodes are arranged between the two conductivity measuring electrodes.

12. The gardening device according to claim 1, wherein the layer consisting of electroless nickel of the two conductivity measuring electrodes is covered with a gold layer.

13. The gardening device according to claim 1, wherein the output device is a communication module for wireless data transmission to an external device.

14. The gardening device according to claim 1, wherein the gardening device has a user interface, e.g. a touchscreen, a selection program of garden plants and/or vegetable species selectable via a user interface, in which the favourable target values (soil moisture, nutrient content in the soil, ambient temperature, lighting conditions in the environment and/or sowing or planting season) for the garden plants and/or vegetable species are stored, as well as a comparator device, in particular a program routine running on the microcontroller, which compares the stored target values for the selected garden plant or vegetable species with the detected actual values for soil moisture, nutrient content in the soil, ambient temperature, ambient light conditions and/or season and transmits the result for output on the output device.

15. A method for sowing or planting, wherein favorable target values for a number of soil properties such as soil moisture, and a nutrient content in the soil, and advantageous ambient temperature, lighting conditions in the environment and/or sowing or planting season are determined for a garden plant and/or vegetable species to be sowed or planted, actual values for the number of soil properties and, advantageously, the ambient temperature, the ambient light conditions and/or the season are determined at a location intended for sowing or planting, then the target values are compared with the actual values and, if the comparison is positive, the sowing or planting is carried out and not otherwise, wherein at least the determination of the actual values, optionally also the determination of the target values, the comparison of the target values with the actual values and the sowing or planting is performed with the aid of a gardening device according to claim 1.
